(19) United States
(12) Patent Application Publication
(10) Pub. No.: US 2006/0084612 A1
(43) Pub. Date: Apr. 20, 2006

(54) Combined therapy against tumors comprising substituted acryloyl distamycin derivatives and protein kinase (serine/threonine kinase) inhibitors

(76) Inventors: Maria Cristina Geroni, Milan (IT); Camilla Fowst, Milan (IT); Paolo Cozzi, Milan (IT)

Correspondence Address: Peter I Bernstein;Scully Scott, Murphy & Presser, 400 Garden City Plaza, Suite 300, Garden City, NY, 11530, US

(21) Appl. No.: 10/500,606

(22) PCT Filed: Dec. 18, 2002
(86) PCT No.: PCT/EP02/13092

§ 371 Date: May 5, 2005

(30) Foreign Application Priority Data
Jan. 2, 2002 (EP) 02075052.7

(51) Int. Cl. A61K 38/08; A61K 38/06

(52) U.S. Cl. 514/17; 514/18

(57) ABSTRACT

The present invention provides the combined use of acryloyl distamycin derivatives, in particular α-bromo- and α-chloro-acryloyl distamycin derivatives of formula (I), as set forth in the specification, and a protein kinase (serine/threonine and tyrosine kinases) inhibitor, in the treatment of tumors. Also provided is the use of the said combinations in the treatment or prevention of metastasis or in the treatment of tumors by inhibition of angiogenesis.

The present invention relates to the field of cancer treatment and provides an antitumor composition comprising a substituted acryloyl distamycin derivative, more particularly an α-bromo- or α-chloro-acryloyl distamycin derivative, and a protein kinase (serine/threonine and tyrosine kinases) inhibitor, having a synergistic antineoplastic effect.

Distamycin A and analogues thereof, hereinafter referred to as distamycin and distamycin-like derivatives, are known in the art as cytotoxic agents useful in antitumor therapy.

Distamycin A is an antibiotic substance with antiviral and antiprotozoal activity, having a polypyrrole framework [Nature 203: 1064 (1964); J. Med. Chem. 32: 774-778 (1989)].

The international patent applications WO 90/11277, WO 98/04524, WO 98/21202, WO 99/50265, WO 99/50266 and WO 01/40181, all in the name of the applicant itself and herewith incorporated by reference, disclose acryloyl distamycin derivatives wherein the amidino moiety of distamycin is optionally replaced by nitrogen-containing ending groups such as, for instance, cyanamidino, N-methylamidino, guanidino, carbamoyl, amidoxime, cyano and the like, and/or wherein the polypyrrole framework of distamycin, or part of it, is replaced by varying carbocyclic or heterocyclic moieties.

The present invention provides, in a first aspect, a pharmaceutical composition for use in antineoplastic therapy in mammals, including humans, comprising a pharmaceutically acceptable carrier or excipient;
an acryloyl distamycin derivative of formula (I): 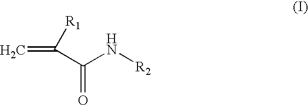
wherein:
R₁ is a bromine or chlorine atom;
R₂ is a distamycin or distamycin-like framework; or a pharmaceutically acceptable salt thereof; and
a protein kinase inhibitor.

The present invention includes, within its scope, the pharmaceutical compositions comprising any of the possible isomers covered by the compounds of formula (I), both considered separately or in admixture, as well as the metabolites and the pharmaceutically acceptable bio-precursors (otherwise known as pro-drugs) of the compounds of formula (I).

In the present description, unless otherwise specified, with the term distamycin or distamycin-like framework R₂ we intend any moiety structurally closely related to distamycin itself, for instance by optionally replacing the ending amidino moiety of distamycin and/or its polypyrrole framework, or part of it, for instance as set forth below.

Protein kinases, hereinafter shrortly referred to as PKs, are a large family of homologous proteins [see, for a reference, J. Clin. Invest. 105: 3 (2000); Cancer Chemotherapy and Biological Response Modifiers, Annual 19 Chapter 11, 236 (2001)].

PKs, as components of signal transduction pathways, play a central role in diverse biological processes such as control of cell growth, metabolism, differentiation, and apoptosis. The development of selective PK inhibitors that can block or modulate diseases with defects in these signaling pathways, has been considered as a promising approach for the development of new anticancer drugs. A selection of these agents is shown in Table 1.

TABLE 1
Low Molecular weight ATP-competitive protein
kinase inhibitors in clinical development

| Target Kinase | Name |
|---|---|
| Bcr-Abl | STI571 (Gleevec; Imatinib) |
| EGF-R | ZD-1839 (Iressa) |
| | OSI-774 (Tarceva) |
| | PKI 166 |
| | EKB-569 |
| | GW572016 |
| PKC/Trk | CEP 2563 |
| PKC | UCN-01 |
| | GCP 41251 (STI 412) |
| | Safingol |
| | Perifosine |
| VEGF-R | SU 5416 (Semaxanib) |
| | CGP 79787 |
| | CP-564959 |
| | ZD 6474 |
| | ZD 2171 |
| | SU-11248 |
| CDKs | Flavopiridol |
| | CI-202 |

The compositions of the invention may be thus comprised by the aforementioned acryloyl distamycin derivative of formula (I) and a protein kinase inhibitor, as listed in table 1.

According to a preferred embodiment of the invention, the PKs inhibitor is selected from STI571 (Gleevec; Imatinib—inhibitor of Bcr-Abl tyrosine kinase), ZD-1839 (Iressa—inhibitor of epidermal growth factor receptor 1 tyrosine kinase), OSI-774 (Tarceva—inhibitor of epidermal growth factor receptor 1 tyrosine kinase) and SU 5416 (Semaxanib—tyrosine kinase inhibitor that inhibits three distinct growth factor receptor targets).

According to another preferred embodiment of the invention, herewith provided are the above pharmaceutical compositions wherein, within the acryloyl distamycin derivative of formula (I), R₁ has the above reported meanings and R₂ is a group of formula (II) below: 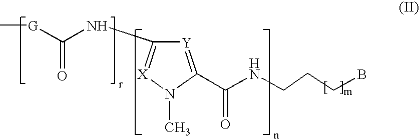
wherein
m is an integer from 0 to 2;
n is an integer from 2 to 5;
r is 0 or 1;
X and Y are, the same or different and independently for each heterocyclic ring, a nitrogen atom or a CH group;
G is phenylene, a 5 or 6 membered saturated or unsaturated heterocyclic ring with from 1 to 3 heteroatoms selected among N, O or S, or it is a group of formula (III) below: 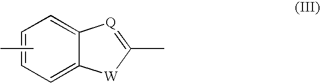
wherein Q is a nitrogen atom or a CH group and W is an oxygen or sulfur atom or it is a group NR₃ wherein R₃ is hydrogen or C₁-C₄ alkyl;
B is selected from the group consisting of 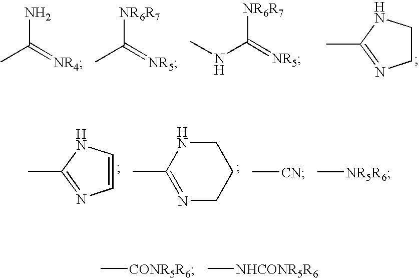
wherein R₄ is cyano, amino, hydroxy or C₁-C₄ alkoxy; R₅, R₆ and R₇, the same or different, are hydrogen or C₁-C₄ alkyl.

In the present description, unless otherwise specified, with the term C₁-C₄ alkyl or alkoxy group we intend a straight or branched group selected from methyl, ethyl, n-propyl, isopropyl, n-butyl, isobutyl, sec-butyl, tert-butyl, methoxy, ethoxy, n-propoxy, isopropoxy, n-butoxy, isobutoxy, sec-butoxy or tert-butoxy.

Preferably, the pharmaceutical compositions of the invention comprise the above acryloyl distamycin derivative of formula (I) wherein R₁ is bromine or chlorine; R₂ is the above group of formula (II) wherein r is 0, m is 0 or 1, n is 4 and B has the above reported meanings.

Still more preferred, within this class, are the pharmaceutical compositions comprising the compounds of formula (I) wherein R₁ is bromine or chlorine; R₂ is the above group of formula (III) wherein r is 0, m is 0 or 1, n is 4, X and Y are both CH groups and B is selected from: 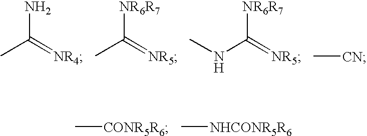
wherein R₄ is cyano or hydroxy and R₅, R₆ and R₇, the same or different, are hydrogen or C₁-C₄ alkyl.

Even more preferred compositions of the invention are those comprising a compound of formula (I) wherein R₁ is bromine, R₂ is the above group of formula (II) wherein r and m are 0, n is 4, X and Y are CH, B is a group of formula: 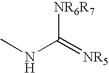
wherein R₅, R₆ and R₇ are hydrogen atoms, optionally in the form of a pharmaceutically acceptable salt thereof.

Pharmaceutically acceptable salts of the compounds of formula (I) are those with pharmaceutically acceptable inorganic or organic acids such as, for instance, hydrochloric, hydrobromic, sulfuric, nitric, acetic, propionic, succinic, malonic, citric, tartaric, methanesulfonic, p-toluenesulfonic acid and the like.

Examples of preferred acryloyl distamycin derivatives of formula (I), within the compositions object of the invention, for instance in the form of pharmaceutically acceptable salts, preferably with hydrochloric acid, are:
1. N-[5-[[[5-[[[2-[(aminoiminomethyl)amino]ethyl]amino]carbonyl]-1-methyl-1H-pyrrol-3-yl]amino]carbonyl]-1-methyl-1H-pyrrol-3-yl]-4-[[[4-[(2-bromo-1-oxo-2-propenyl)amino]-1-methyl-1H-pyrrol-2-yl [carbonyl]amino]-1-methyl-1H-pyrrole-2-carboxamide hydrochloride (Brostallicin);
2. N-(5{[(5-{[95-{[(2-{[amino(imino)methyl]amino}propyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride-
3. N-(5-{[(5-{[(5-{[(3-amino-3-iminopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;
4. N-(5-{[(5-{[(5-{[(3-amino-3-iminopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-imidazole-2-carboxamide hydrochloride;
5. N-(5-{[(5-{[(5-{[(3-amino-3-iminopropyl)amino]carbonyl}-1-methyl-1H-pyrrol-3-yl) amino]carbonyl}-1-methyl-1H-pyrrol-3-yl)amino]carbonyl}-1-methyl-1H-pyrrol-3-yl)-3-[(2-bromoacryloyl)amino]-1-methyl-1H-pyrazole-5-carboxamide hydrochloride;
6. N-(5-{[(5-{[(5-{[(3-amino-3-oxopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-3-[(2-bromoacryloyl)amino]-1-methyl-1H-pyrazole-5-carboxamide;
7. N-(5-{[(5-{[(5-{([(2-{[amino(imino)methyl]amino}ethyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-chloroacryloyl)amino]-1-methyl-1H-pyrrole-2-carboxamide hydrochloride;
8. N-(5-{[(5-{[(3-{[amino(imino)methyl]amino} propyl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;
9. N-(5-{[(5-{[(3-amino-3-iminopropyl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2 carboxamide hydrochloride; and
10. N-{5-[({5-[({5-[({3-[(aminocarbonyl)amino]propyl} amino)carbonyl]-1-methyl-1H-pyrrol-3-yl)}amino)carbonyl]-1-methyl-1H-pyrrol-3-yl}amino)carbonyl]-1-methyl-1H-pyrrol-3-yl}-4-[(2-bromoacryloyl)amino]-1-methyl-1H-pyrrole-2-carboxamide.

The above compounds of formula (I), either specifically identified as such or by means of the general formula, are known or easily prepared according to known methods as reported, for instance, in the aforementioned international patent applications WO 90/11277, WO 98/04524, WO 98/21202, WO 99/50265 and WO 99/50266 as well as in WO 01/40181.

The present invention further provides a product, otherwise referred to as kit of parts, comprising an acryloyl distamycin derivative of formula (I), as defined above, and a PK inhibitor, as a combined preparation for simultaneous, separate or sequential use in antitumor therapy.

A further aspect of the present invention is to provide a method of treating a mammal, including humans, suffering from a neoplastic disease state, which method comprises administering to said mammal the above acryloyl distamycin derivative of formula (I) and a PK inhibitor, in amounts effective to produce a synergistic antineoplastic effect. The present invention also provides a method for lowering the side effects caused by antineoplastic therapy with an antineoplastic agent in a mammal in need thereof, including humans, the method comprising administering to said mammal a combined preparation comprising a PK inhibitor and an acryloyl distamycin derivative of formula (I), as defined above, in amounts effective to produce a synergistic antineoplastic effect.

By the term “synergistic antineoplastic effect”, as used herein, it is meant the inhibition of the growth tumor, preferably the complete regression of the tumor, by administering an effective amount of the combination comprising an acryloyl distamycin derivative of formula (I) and a PK inhibitor to mammals, including humans.

By the term “administered ” or “administering”, as used herein, it is meant parenteral and/or oral administration; the term “parenteral” means intravenous, subcutaneous and intramuscular administration.

In the method of the present invention, the acryloyl distamycin derivative may be administered simultaneously with the PK inhibitor or, alternatively, both compounds may be administered sequentially in either order.

In this respect, it will be appreciated that the actual preferred method and order of administration will vary according to, inter alia, the particular formulation of the acryloyl distamycin of formula (I) being used, the particular formulation of the PK inhibitor being used, the particular tumor model being treated as well as the particular host being treated.

To administer the acryloyl distamycin derivative of formula (I), according to the method of the invention, the course of therapy generally employed comprises doses varying from about 0.05 to about 100 mg/m² of body surface area and, more preferably, from about 0.1 to about 50 mg/m² of body surface area.

For the administration of the PK inhibitor, according to the method of the invention, the course of therapy generally employed may be as follows.

For the administration of STI571 (Imatinib), doses varying from about 5 mg/day to about 5000 mg/day and, more preferably, from about 30 to about 1000 mg/day.

For the administration of ZD 1839 (Iressa) doses varying from about 5 mg/day to about 10000 mg/day and, more preferably, from about 50 to about 1000 mg/day.

For the administration of OSI-774 (Tarceva) doses varying from about 5 mg/day to about 10000 mg/day and, more preferably, from about 50 to about 1000 mg/day.

For the administration of SU 5416 (Semaxanib) doses varying from about 1 mg/m² to about 1000 mg/m² of body surface area and, more preferably, from about 10 to about 500 mg/m² of body surface area.

The antineoplastic therapy of the present invention is particularly suitable for treating breast, ovary, lung, colon, kidney, stomach, pancreas, liver, melanoma, leukemia and brain tumors in mammals, including humans.

In a further aspect, the present invention is directed to a pharmaceutical composition comprising an effective amount of an acryloyl distamycin derivative of formula (I), as defined above, and a PK inhibitor, in the preparation of a medicament for use in the prevention or treatment of metastasis or in the treatment of tumors by inhibition of angiogenesis.

As the effect of an acryloyl distamycin derivative of formula (I) and a PK inhibitor is significantly increased without a parallel increase of toxicity, the combined therapy of the present invention enhances the antitumoral effects of the acryloyl distamycin derivative and of the PK inhibitor and, hence, provides the most effective and least toxic treatment for tumors.

1. A pharmaceutical composition comprising a pharmaceutically acceptable carrier or excipient and, as active ingredient,
an acryloyl distamycin derivative of formula (I): 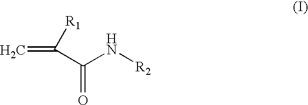
wherein:
R₁ is a bromine or chlorine atom;
R₂ is a distamycin or distamycin-like framework; or a pharmaceutically acceptable salt thereof; and
a protein kinase inhibitor.

2. A pharmaceutical composition according to claim 1 wherein the protein kinase inhibitor is selected from the group consisting of ST1571, ZD-1839, OSI-774, PKI 166, EKB-569, GW572016, CEP 2563, UCN-01, GCP 41251 (STI 412), Safingol, Perifosine, SU 5416, CGP 79787, CP-564959, ZD 6474, ZD 2171, SU-11248, Flavopiridol, and CI-202.

3. A pharmaceutical composition according to claim 2 wherein the protein kinase inhibitor is selected from the group consisting of STI571, ZD-1839, OSI-774 and SU 5416.

4. A pharmaceutical composition according to claim 1 comprising an acryloyl distamycin derivative of formula (I) 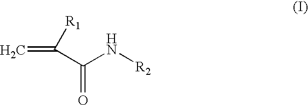
wherein:
R₁ is a bromine or chlorine atom;
R₂ is a group of formula (II) 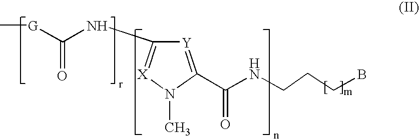
wherein
m is an integer from 0 to 2;
n is an integer from 2 to 5;
r is 0 or 1;
X and Y are, the same or different and independently for each heterocyclic ring, a nitrogen atom or a CH group;
G is phenylene, a 5 or 6 membered saturated or unsaturated heterocyclic ring with from 1 to 3 heteroatoms selected among N, O or S, or it is a group of formula (III) below: 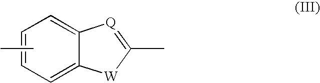
wherein Q is a nitrogen atom or a CH group and W is an oxygen or sulfur atom or it is a group NR₃ wherein R₃ is hydrogen or C₁-C₄ alkyl;
B is selected from the group consisting of 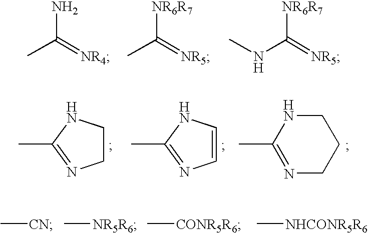
wherein R₄ is cyano, amino, hydroxy or C₁-C₄ alkoxy; R₅, R₆ and R₇, the same or different, are hydrogen or C₁-C₄ alkyl.

5. A pharmaceutical composition according to claim 4 comprising an acryloyl distamycin derivative of formula (I) wherein R₁ and R₂ are as defined in claim 4, r is 0, m is 0 or 1, n is 4, X and Y are both CH groups and B is selected from: 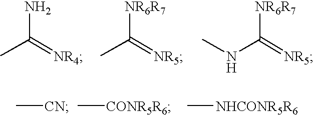
wherein P₄ is cyano or hydroxy and R₅, R and R₇, the same or different, are hydrogen or C₁-C₄ alkyl.

6. A pharmaceutical composition according to claim 5 comprising an acryloyl distamycin derivative of formula (I) wherein R₁ is bromine, R₂ is a group of formula (II) wherein r and m are 0, n is 4, X and Y are CH, B is a group of formula 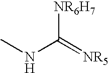
wherein R₅, R₆ and R₇ are hydrogen atoms, optionally in the form of a pharmaceutically acceptable salt.

7. A pharmaceutical composition according to claim 1 comprising an acryloyl distamycin derivative, optionally in the form of a pharmaceutically acceptable salt, selected from the group consisting of:
1. N-[5-[[[5-[[[2-[(aminoiminomethyl)amino]ethyl]amino]carbonyl]-1-methyl-1H-pyrrol-3-yl]amino]carbonyl]-1-methyl-1H-pyrrol-3-yl]-4-[[[4-[(2-bromo-1-oxo-2-propenyl)amino]-1-methyl-1H-pyrrol-2-yl[carbonyl)amino]-1-methyl-1H-pyrrole-2-carboxamide hydrochloride (Brostallicin);
2. N-(5-{[(5-{[(5-{[(2-{[amino(imino)methyl]amino}propyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;
3. N-(5-{[(5{[(5-{[(3-amino-3-iminopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;
4. N-(5-{[(5-{[(5-{[(3-amino-3-iminopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-imidazole-2-carboxamide hydrochloride;
5. N-(5-{[(5-{[(5-{[(3-amino-3-iminopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-3-[(2-bromoacryloyl)amino)-1-methyl-1H-pyrazole-5-carboxamide hydrochloride;
6. N-(5-{[(5-{[(5-{[(3-amino-3-oxopropyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-3-[(2-bromoacryloyl)amino]-1-methyl-1H-pyrazole-5-carboxamide;
7. N-(5-{[(5-{[(5-{[(2-{[amino(imino)methyl]amino} ethyl)amino]carbonyl}-1-methyl-1H-pyrrol-3-yl)amino]carbonyl}-1-methyl-1H-pyrrol-3-yl)amino]carbonyl}-1-methyl-1H-pyrrol-3-yl)-4-[(2-chloroacryloyl)amino]-1-methyl-1H-pyrrole-2-carboxamide hydrochloride;
8. N-(5-{[(5-{[(3-{[amino(imino)methyl]amino}propyl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride;
9. N-(5-{[(5-{[(3-amino-3-iminopropyl)amino]carbonyl}-1-methyl-1H-pyrrol-3-yl)amino]carbonyl}-1-methyl-1H-pyrrol-3-yl)-4-[(2-bromoacryloyl)amino]-1-methyl-1H-pyrrole-2-carboxamide hydrochloride; and
10. N-{5-[({5-[({5-[({3-[(aminocarbonyl)amino]propyl} amino)carbonyl]-1-methyl-1H-pyrrol-3-yl} amino)carbonyl]-1-methyl-1H-pyrrol-3-yl} amino)carbonyl]-1-methyl-1H-pyrrol-3-yl}-4-[(2-bromoacryloyl)amino]-1-methyl-1H-pyrrole-2-carboxamide.

8. A pharmaceutical composition comprising a pharmaceutically acceptable carrier or excipient and, as active ingredient,
N-[5-[[[5-[[[2-[(aminoiminomethyl)amino]ethyl]amino]carbonyl]-1-methyl-1H-pyrrol-3-yl]amino]carbonyl]-1-methyl-1H-pyrrol-3-yl]-4-[[[4-[(2-bromo-1-oxo-2-propenyl)amino])-1-methyl-1H-pyrrol-2-yl[carbonyl]amino]-1-methyl-1H-pyrrole-2-carboxamide hydrochloride (Brostallicin); and
a protein kinase inhibitor selected from the group consisting of STI571, ZD-1839, OSI-774, and SU 5416.

9. Products comprising an acryloyl distamycin derivative of formula (I): 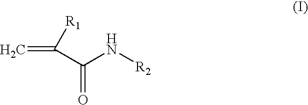
wherein:
R₁ is a bromine or chlorine atom;
R₂ is a distamycin or distamycin-like framework; or a pharmaceutically acceptable salt thereof; and a protein kinase inhibitor, as a combined preparation for simultaneous, separate or sequential use in the treatment of tumors.

10. Products according to claim 9 wherein the protein kinase inhibitor is selected from the group consisting of STI571, ZD-1839, OSI-774, PKI 166, EKB-569, GW572016, CEP 2563, UCN-01, GCP 41251 (STI 412), Safingol, Perifosine, SU 5416, CGP 79787, CP-564959, ZD 6474, ZD 2171, SU-11248, Flavopiridol, and CI-202.

11. Products according to claim 10 wherein the protein kinase inhibitor is selected from the group consisting of STI571, ZD-1839, OSI-774 and SU 5416.

12. Products according to claim 9 comprising an acryloyl distamycin derivative of formula (I) 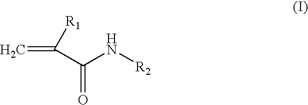
wherein:
R₁ is a bromine or chlorine atom;
R₂ is a group of formula (II) 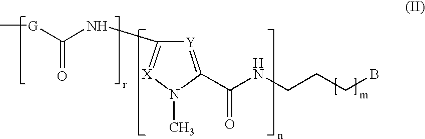
wherein
m is an integer from 0 to 2;
n is an integer from 2 to 5;
r is 0 or 1;
X and Y are, the same or different and independently for each heterocyclic ring, a nitrogen atom or a CH group;
G is phenylene, a 5 or 6 membered saturated or unsaturated heterocyclic ring with from 1 to 3 heteroatoms selected among N, O or S, or it is a group of formula (III) below: 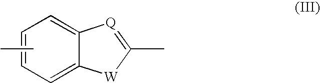
wherein Q is a nitrogen atom or a CH group and W is an oxygen or sulfur atom or it is a group NR₃ wherein R₃ is hydrogen or C₁-C₄ alkyl;
B is selected from the group consisting of 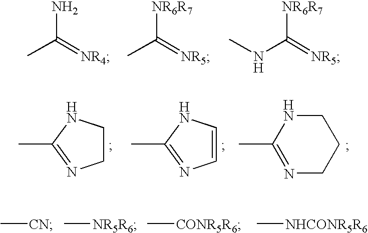
wherein R₄ is cyano, amino, hydroxy or C₁-C₄ alkoxy; R₅, R₆ and R₇, the same or different, are hydrogen or C₁-C₄ alkyl.

13. Products according to claim 9 comprising an acryloyl distamycin derivative of formula (I) wherein R₁ is bromine, R₂ is a group of formula (II) wherein r and m are 0, n is 4, X and Y are CH, B is a group of formula 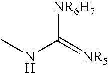
wherein R₅, and R₇ are hydrogen atoms, optionally in the form of a pharmaceutically acceptable salt.

14. Products according to claim 9 wherein the acryloyl distamycin derivative is selected from the group as defined in claim 7.

15. Products comprising the acryloyl distamycin derivative N-[5-[[[5-[[[2-[(aminoiminomethyl)amino]ethyl]amino]carbonyl]-1-methyl-1H-pyrrol-3-yl]amino]carbonyl]-1-methyl-1H-pyrrol-3-yl]-4-[[[4-[(2-bromo-1-oxo-2-propenyl)amino]-1-methyl-1H-pyrrol-2-yl[carbonyl]amino]-1-methyl-1H-pyrrole-2-carboxamide hydrochloride (Brostallicin), and a protein kinase inhibitor selected from the group consisting of STI571, ZD-1839, OSI-774, and SU 5416; as a combined preparation for simultaneous, separate or sequential use in the treatment of tumors.

16. Use of an acryloyl distamycin derivative of formula (I), as defined in claim 1, in the preparation of a medicament to be used in combination therapy with a protein kinase inhibitor, in the treatment of tumors.

17. Use according to claim 16 wherein the medicament further comprises the said protein kinase inhibitor.

18. Use according to claims 16 or 17 wherein the protein kinase inhibitor is as defined in claim 2.

19. Use according to claims 16 or 17 wherein the acryloyl distamycin derivative is selected from the group as defined in claim 7.

20. Use of the acryloyl distamycin derivative N-[5-[[[5-[[[2-[(aminoiminomethyl)amino]ethyl]amino]carbonyl]-1-methyl-1H-pyrrol-3-yl]amino]carbonyl]-1-methyl-1H-pyrrol-3-yl]-4-[[[4-[(2-bromo-1-oxo-2-propenyl)amino]-1-methyl-1H-pyrrol-2-yl[carbonyl]amino]-1-methyl-1H-pyrrole-2-carboxamide hydrochloride (Brostallicin), in the preparation of a medicament to be used in combination therapy with a protein kinase inhibitor selected from the group consisting of STI571, ZD-1839, OSI-774, and SU 5416, in the treatment of tumors.

21. Use according to any one of claims from 16 to 20 wherein the tumor is selected from breast, ovary, lung, colon, kidney, stomach, pancreas, liver, melanoma, leukemia and brain tumors.

22. Use of an acryloyl distamycin derivative of formula (I), as defined in claim 1, in the preparation of a medicament to be used in combination therapy with a protein kinase inhibitor, in the prevention or treatment of metastasis or in the treatment of tumors by inhibition of angiogenesis.

23. Use according to claim 22 wherein the medicament further comprises the said protein kinase inhibitor.

24. A method of treating a mammal, including humans, suffering from a neoplastic disease state, which method comprises administering to said mammal the acryloyl distamycin derivative of formula (I), as defined in claim 1, and a protein kinase inhibitor, in amounts effective to produce a synergistic antineoplastic effect.

25. A method according to claim 24 wherein the acryloyl distamycin derivative is N-[5-[[[5-[[[2-[(aminoiminomethyl)amino]ethyl]amino]carbonyl) 1-methyl-1H-pyrrol-3-yl]amino]carbonyl]-1-methyl-1H-pyrrol-3-yl]-4-[[[4-[(2-bromo-1-oxo-2propenyl)amino]-1-methyl-1H-pyrrol-2-yl[carbonyl]amino]-1-methyl-1H-pyrrole-2-carboxamide hydrochloride (Brostallicin), and the protein kinase inhibitor is selected from the group consisting of STI571, ZD-1839, OSI-774, and SU 5416.

26. A method for lowering the side effects caused by antineoplastic therapy with an antineoplastic agent, in a mammal in need thereof including humans, the method comprising administering to said mammal a combined preparation comprising a protein kinase inhibitor and an acryloyl distamycin derivative of formula (I), as defined in claim 1, in amounts effective to produce a synergistic antineoplastic effect.

27. A method according to claim 26 wherein the acryloyl distamycin derivative is N-[5-[[[5-[[[2-[(aminoiminomethyl)amino]ethyl]amino]carbonyl-1-methyl-1H-pyrrol-3-yl]amino]carbonyl]-1-methyl-1H-pyrrol-3-yl]-4-[[[4-[(2-bromo-1-oxo-2-propenyl)amino]-1-methyl-1H-pyrrol-2-yl[carbonyl]amino]-1-methyl-1H-pyrrole-2-carboxamide hydrochloride (Brostallicin), and the protein kinase inhibitor is selected from the group consisting of STI571, ZD-1839, OSI-774, and SU 5416.
